(19) United States
(12) Patent Application Publication
(10) Pub. No.: US 2007/0100166 A1
(43) Pub. Date: May 3, 2007

(54) Catalyst and process for the preparation of unsymmetrical ketones

(76) Inventors: William A. Beavers, Longview, TX (US); Alexey V. Ignatchenko, Longview, TX (US); Zhufang Liu, Kingsport, TN (US); Carey D. Ashcroft, Longview, TX (US); Tracy M. White, Longview, TX (US)

Correspondence Address: BUCHANAN, INGERSOLL & ROONEY PC, POST OFFICE BOX 1404, ALEXANDRIA, VA, 22313-1404, US

(21) Appl. No.: 11/525,150

(22) Filed: Sep. 22, 2006

Related U.S. Application Data
(60) Provisional application No. 60/719,872, filed on Sep. 23, 2005.

(51) Int. Cl. C07C 45/48

(52) U.S. Cl. 568/397

(57) ABSTRACT

Carboxylic acid mixtures form unsymmetrical ketones in yields approaching statistical using zirconia catalysts promoted with Group IA and IIA elements. Active catalysts exist in their monoclinic or tetragonal but not cubic form. And the level of promoter loading is generally less than ten percent. The advantages of this catalyst over other ketonization catalysts include its high selectivity to ketones, its low formation of dehydrogenated byproducts, and its stability. The catalyst stability permits its regeneration to remove carbon accumulations by air oxidation. This regeneration restores full catalytic activity.

### CROSS-REFERENCE TO RELATED APPLICATIONS

This application claims the benefit of the filing date of U.S. Provisional Patent Application No. 60/719,872, filed Sept. 23, 2005; the entire content of which is hereby incorporated by reference.

### FIELD OF THE INVENTION

Preparation and use in a process of a rugged catalyst for the manufacture of unsymmetrical ketones from mixtures of carboxylic acids. The specific unsymmetrical ketone of interest is methyl isopropyl ketone from mixtures of acetic and isobutyric acids.

### BACKGROUND OF THE INVENTION

The preparation of ketones from carboxylic acids has been known for more than a century. It takes place according to the following equation so that it is effectively a decarboxylative dehydration:

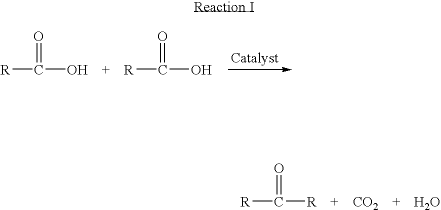

Calling the reaction a ketonic decarboxylation, March cites thorium, iron, barium, and calcium as catalysts. Hussman reveals a more exhaustive list of catalysts including metal ions and metal oxides containing lithium, sodium, zinc, cadmium, magnesium, beryllium, gallium, indium, tin, titanium, zirconium, chromium, manganese, and cerium. Glinski et al. extend this list to include vanadium, bismuth, nickel, aluminum, copper, lead, cobalt, neodynium, and lanthanum. These lists include members from virtually every family of the alkalai, alkaline earth, and transition metals and several examples of lanthanide and actinide elements.

The pathways leading from the carboxylic acids to ketones are numerous. As summarized by Rajadurai, the pathways include mechanisms involving anhydrides, beta-keto acids, carbonium ions, ketenes, adsorbed carboxylic acids, and carboxylate ion formation for substrates having at least one hydrogen on the carbon adjacent to the carboxyl group and concerted mechanisms for those substrates lacking hydrogens on the alpha-carbon. Differences are displayed in each catalyst acting on each substrate and will change with changing temperatures.

Each catalyst displays its own characteristic activity with associated strengths and weaknesses. These include efficiencies for preparing symmetrical ketones, selectivities for unsymmetrical ketones, aldehyde formation (as a special class of ketones in which one of the R groups is hydrogen), catalyst lifetimes, and catalyst stabilities. Even the physical states of the catalyst and reactants relate to these strengths and weaknesses. Therefore no single catalyst is superior for all applications.

Of particular interest in this regard because of their high lattice energies and acidities are the group IVB elements. At least one member, titania, appears particularly adept at catalyzing the preparation of unsymmetrical ketones according to Schommer et al. The base element promoters presumably modify the detrimental acidity of the titania. Moreover their starting materials include carboxylic acids and/or ketones.

However the primary use for titania as a pigment stems largely from its limited structural strength which impairs its catalytic applications. Furthermore titania exhibits oxidation-reduction properties at high temperatures leading to unsaturated by-products which are nearly impossible to remove without corrective re-hydrogenation in separate hydrogenation facilities.

Structurally more rugged is zirconia. Its cubic form although catalytically inactive is among the hardest in nature. And its monoclinic and tetragonal forms are almost as durable.

The ability of these latter forms to catalyze the dehydrative decarboxylation of carboxylic acids was shown in Japanese Kokai patent JP 57-197,237 in which propionic acid was converted into diethyl ketone in nearly quantitative selectivity at nearly quantitative conversion. And the high lattice energy of zirconia accompanying this structural strength also coincidentally limits the extent of oxidation-reduction activity so that the unsaturated by-products plaguing titania catalysts are limited obviating the need for a polishing hydrogenation.

Parida and Mishra found basic modifiers which enhanced the catalytic activity. But the application still remained suitable exclusively for symmetrical ketones. Their catalysts proved highly effective in converting acetic acid into acetone. But unsymmetrical ketones remained ellusive.

### SUMMARY OF THE INVENTION

We unexpectedly discovered an improved heterogeneous zirconia catalyst leading to unsymmetrical ketones from mixtures of carboxylic acids. The improvement manifests itself only at atypical conditions compared with untreated zirconia. And the catalyst consists of moderate to high surface area zirconia treated with group Ia and group IIa metal hydroxides, oxides, or materials which become hydroxides or oxides under the reaction conditions.

A key feature of this catalyst is its stability which permits its operation at the atypical, most preferred conditions for protracted times without loss of activity. Furthermore when deactivation occurs, generally by a coking mechanism, the stability of this catalyst permits regeneration simply by passing air diluted with an inert diluent including nitrogen, water, and carbon dioxide, for sufficient time to burn away the carbon residue thereby restoring nearly complete activity. Under these reaction conditions, this catalyst will produce unsymmetrical ketones from mixtures of carboxylic acid substrates in amounts approaching statistical.

### DETAILED DESCRIPTION OF THE INVENTION

Besides the ability to form ketones from carboxylic acids, the most important property of the catalyst in this invention is its stability. In its most stable form, cubic zirconia is catalytically inert. Including group Ia and IIa elements in the catalyst stabilize the catalytic tetragonal and especially its octahedral forms thereby preserving its catalytic activity.

1. Base Modified Catalysts

The base modification can occur by contacting the zirconia catalyst with the metal salt which is either basic itself or becomes basic under the reaction conditions. Favorable metals include sodium, potassium, cesium, and lithium from group Ia and calcium, strontium, barium, and magnesium from group IIa. The other members of Groups Ia and IIa also produce ketones in the same manner, but they are generally less effective. The more desirable of these promoters include potassium, sodium, rubidium, magnesium, calcium, strontium, and barium. And the most desirable elements include sodium, potassium, and calcium.

Suitable basic counterions include hydroxide, carbonate, and oxide. Ones which become basic under the reaction conditions either by oxidation or pyrolysis include bicarbonate, carboxylate salts of mono- or poly-basic carboxylic acids containing 1-20 carbon atoms, nitrate, nitrite, or any of various organometallics which under calcining conditions oxidize to hydroxides and oxides.

Incorporating the Group Ia or IIa promoter can take place by several methods. The first is an exchange effected by soaking a solution of the exchanging agent in a suitable solvent with the solid zirconium catalyst. The second is by incipient wetness techniques with any amount of exchanging agent. Other methods include co-precipitation of zirconia from a suitable precursor and the promoter simultaneously.

In any case there is a maximum amount of exchanging agent which is optimum. The production of ketones will take place at levels above or below the optimum; however, the production of ketones, especially mixed ketones, will not be optimal at these levels.

The optimum level of catalyst promoter depends on the exact agent. But with an agent such as potassium hydroxide, it will typically fall in the 0.1-20 weight percent range. More desirable levels are found in the 0.25-10 weight percent range. And the most desired loading level is 0.5-5 weight percent.

The preparation of the zirconia itself is also standard for those well versed in the art. Thus hydrolysis of zirconium oxychloride or zirconium tetrachloride with aqueous sodium hydroxide at ambient temperatures to near the boiling point of water followed by washing with distilled or deionized water till sodium and chloride ions no longer are present is the most facile method. Instead of the zirconium chlorides, zirconium (IV) alkoxides in which the alkoxy groups contain 1-20 carbon atoms each provide an equally suitable substitute for this same treatment. In all cases the resulting material may or may not be calcined at various temperatures for various lengths of time. The calcining treatment largely determines the surface area of the untreated zirconia and its accompanying activity. The activity of the overall catalyst generally parallels the surface area of the zirconia.

2. Ketone Production from Carboxylic Acids

Untreated zirconia catalyst displays a high lattice energy which the base treatment helps lower. In its reaction with carboxylic acids high lattice energy translates into a very discriminating catalyst which remains relatively high despite the base treatment. The temperatures at which ketones can form are moderate. But at these temperatures the high lattice energy renders it very discriminating toward those carboxylic acids with which it will react thereby limiting the types of products formed. Electronic and resonance effects, but especially steric effects control the degree of interaction and reaction of the individual carboxylic acid with the catalyst surface.

a. Temperatures

The strategy for making mixed ketones is to operate at temperatures beyond those permitting discrimination. Compared with untreated zirconia, the full benefit of the group IA and IIA promoter treatment becomes apparent only under these conditions for unknown reasons. At sub-optimum temperatures the untreated zirconia is actually the better catalyst according to its selectivity to unsymmetrical ketone products.

The temperatures in the reactive zone preferably are in the 250-700° C. range. More preferably they exist in the 350-600° C. range. And most preferably they occur in the 450-500° C. range. The full benefit of the promoted zirconia manifests itself only under the most preferable conditions. At all other preferable temperatures the reaction rate for the promoted zirconia exceeds that for the untreated zirconia. But side reactions at these conditions are also faster so that the enhanced selectivity to the desired unsymmetrical ketone is not exhibited.

At the most preferable temperatures the reacting carboxylic acids become energetic enough to react indiscriminately with the catalyst surface. The base treatment accelerates the reaction by increasing the catalyst susceptibility to react with the carboxylic acids. The electronic, resonance, and steric effects become less significant compared with the energy available to force the reaction. Therefore overcoming its ability to discriminate, the catalyst produces ketones in a ratio approaching statistical. The production of symmetrical and mixed ketones approaches what one expects based on the molar ratio of the starting acids.

b. Feed Rates

Important also is the rate at which the substrates are fed. Many ketonization reactions exist at temperatures above the boiling points of the substrates so that the reactions take place in the gas phase. Since this is not a requirement and to avoid confusion, feed rates are understood to refer to the quantity of condensed substrate fed through the system regardless of what form they actually exist in the reaction zone.

The optimum feed rate varies directly with the temperature with higher feed rates accompanying the higher temperatures. This feed rate will usually fall in the range of 0.1 to 100 volumes of condensed substrate per volume of catalyst per hour. The most preferable feed rates are chosen to minimize the amount of unreacted substrates without pushing the reaction to such extremes that side reactions begin to dominate. As such the conversion of the least reactive acid is desirably 85-99 percent. A more desirable range is 90-98 percent. And the most desirable conversion of starting acids is 95-97 percent. Although the reaction will take place beyond these limits, below 85 percent conversion will give outstanding overall ketone selectivity with fewer by-products, but will require an additional, more costly distillation during product recovery to separate the unreacted starting material for recycle into product. And conversions beyond 99 percent begin to entail significant product losses as increasing contributions by side reactions convert already formed product as well as the starting materials into side products.

At these conditions, untreated zirconia also produces mixed ketones albeit with limited selectivities. Undergoing the group Ia or IIa treatment raises the production of mixed ketones significantly approaching those statistically expected. This increased mixed ketone production coupled with increasing the stability of catalytically active monoclinic and tetragonal phases produce a clearly superior catalyst.

c. Feed Ratios

Also important for the success of this reaction is to use the proper ratio of the starting materials. The stoichiometry of mixed ketone preparation required a molar ratio of 1:1 of the starting carboxylic acids to achieve the maximum amount of the mixed product while minimizing the production of the two symmetrical ketones. In reality one of the starting acids might be more expendable than the other so that by using more of the more expendable acid, the yield of unsymmetrical ketone product from the less expendable acid increases. Similar arguments apply to one or the other symmetrical ketone products being expendable is understood in terms of costliness or availability.

Although the catalyst in this invention does not produce statistical quantities of all components, the statistically expected production serves as a guideline for what the catalyst can produce. The following table includes expected product ratios as well as calculated yields based on the starting materials for the indicated equation:
A-COOH+B—COOH→A2C═O+A-(C═O)—B+B2C═O

TABLE 1
Statistical Limits of Unsymmetrical Ketones Produced from
Different Ratios of Starting Carboxylic Acids

| Molar | | | | | |
|---|---|---|---|---|---|
| Ratios | | | | | |
| A- | | | | A-(C═O)-B Yield | |
| COOH: | Statistical Product Mix | | | Based On - | |
| B- | (Mole %) | | | (Mole %) | |
| COOH | A2C═O | A-(C═O)-B | B2C═O | A-COOH | B-COOH |
| 0.5:1 | 11.2 | 44.4 | 44.4 | 80.0 | 50.0 |
| 1:1 | 25.0 | 50.0 | 25.0 | 66.7 | 66.7 |
| 1.25:1 | 30.9 | 49.4 | 19.7 | 61.5 | 71.4 |
| 1.5:1 | 36.0 | 48.0 | 16.0 | 57.1 | 75.0 |
| 1.75:1 | 40.5 | 46.3 | 13.2 | 53.3 | 77.8 |
| 2:1 | 44.4 | 44.4 | 11.2 | 50.0 | 80.0 |
| 3:1 | 56.3 | 37.5 | 6.2 | 40.0 | 85.7 |
| 4:1 | 64.0 | 32.0 | 4.0 | 33.3 | 88.9 |
| 5:1 | 69.4 | 27.8 | 2.8 | 28.6 | 90.9 |

The choice of which ratio of starting materials to use will depend on the overall objectives, the deviation of the actual catalyst from these statistical limits, and what to do with the by-products. Evident from this table at the higher ratios of starting materials is that the small amount of the one by-product formed (B2C═O) will result in an abundance of the other by-product (A2C═O).

For this reason the preferred ratio of starting carboxylic acids is generally in the 5:1 to 1:1 range with the material of less importance being in abundance. A more preferable range to optimize the return without co-producing large amounts of by-product is 3:1 to 1:1. And the most preferable range of starting carboxylic acids is 2:1 to 1:1. In the latter case the selectivity to the unsymmetrical ketone is good without producing unacceptably large amounts of by-product.

3. Catalyst Regeneration

A primary manifestation of this superiority occurs during the catalyst regeneration. At the lower temperature ranges for which this catalyst produces ketones, its production of the mixed ketones is below that statistically expected attributable to the strong discriminating effect it displays in reacting with different sized substrates. At the higher temperature ranges the catalyst efficiency falters because the catalyst surface becomes covered with carbon arising from side reactions. This so-called coking process slows down and eventually stops ketone production completely as the catalytically active sites becomes increasingly clogged with inert carbon.

The ability to regenerate the activity by removing the carbon blockage proves crucial. Therefore the catalyst stability is critical. This oxidative regeneration without conversion of the catalyst into its inert forms is obvious.

This regenerability provides another advantage over other Group IVB catalysts. Titania converts from the catalytically active but metastable anatase phase into the most stable but catalytically inactive rutile phase at temperatures of 200-900° C. depending on the acidity of the environment. Therefore oxidative regeneration of titania catalysts leads to loss in activity under typical regenerative conditions.

The strategy for the promoted zirconia catalyst preferably uses 0.1-100 percent oxygen at appropriate temperatures for various times the key being how much carbon dioxide and carbon monoxide exist in the off-gases. A more preferable range is 1-20 percent with the most preferable range being 3-10 percent. Any inert diluent is acceptable including nitrogen, helium, argon, neon, and water. An interesting strategy is to use carbon dioxide as the oxidant monitoring the amount of carbon monoxide existing in the off-gases. The carbon dioxide serves as both the inert diluent and the source of oxygen. And it may be diluted with any mixture of other inert diluents itself. This carbon dioxide strategy generally requires higher regeneration temperatures.

The optimum regeneration temperatures fall in the 300-700° C. range. More preferably they exist in the 350-600° C. range. And the most preferable temperatures for catalyst regeneration are 400-500° C. You will note these are the same temperatures at which the ketonization reaction takes place albeit in the absence of the regenerating oxidant. At the most preferable regeneration temperatures, the time required to reduce the carbon oxides to 1 percent of their highest level is generally 0.5 to 8 hours with a feed rate of 10 catalyst volumes per hour of the regenerating gas.

This treatment removes up to several weight percent carbon on the catalyst surface. It also restores essentially complete catalyst activity. The catalyst integrity is unaffected because of the inherent strength of the zirconia material and the fact that the treatment takes place at mild temperatures.

Suitable inert agents to use during the regeneration process include water, nitrogen, carbon dioxide, argon, helium, or neon. The most preferred agents are water and nitrogen solely because they are most readily available and least expensive.

4. Nature of the Catalyst

This discussion helps to understand the features of this invention without necessarily binding the authors to any theory. It is understood that the explanations are merely consistent with these results without limiting their utility or efficacy.

The catalyst surface consists of closely packed active sites of hydrous zirconia of the general formula, ZrO(OH)2, on which sites the carboxylic acids condense. This process is aided by the Group Ia and Group IIa metal hydroxides which combine with the bulk of the catalyst to form basic sites on which the organic acids can react. More importantly these metal hydroxides catalyze the formation of the active hydrous zirconia from tightly bound and therefore poorly reactive and hydrophobic bulk ZrO2 according to the following equations, 1 and 2:
ZrO2+MOH→ZrO(OH)O⁻M⁺ 1.
ZrO(OH)O⁻M⁺+H2O⇄ZrO(OH)2+MOH 2.

Note the presence of the active hydrous zirconia, the free metal hydroxide, and the metal hydroxide-zirconia derivatives at equilibrium in this catalyst. It is thought the reaction of the organic carboxylic acid takes place with all components simultaneously, albeit at different rates, by the following possible reaction sequence (equations 3-6). It is understood this representation merely supports the actual chemistry of the catalyst which might take place by entirely different mechanisms altogether without effect on the efficacy of the overall process:
ZrO(OH)O⁻M⁺+RCO2H→ZrO(O2CR)O^{-M}⁺+H2O 3.

In equation 3, the acid proton initially coordinates or hydrogen bonds with the negatively charged zirconia oxygen in close proximity to the adjacent hydroxyl group. A rapid shift of this proton to that adjacent hydroxyl group forms coordinated water which readily splits out at the high reaction temperature. This required breaking of the strong O-Zr bond is the primary reason high lattice energies impede this reaction. The resulting positively charged vacancy provides a slot into which the negatively charged carboxylate anion, existing as an ion pair, can combine giving the intermediate shown.

A repetition of this reaction with a second carboxylic acid provides a dicarboxylate derivative in equation 4. This equation represents the chemical species which holds the two reacting carboxylic acids together in the proper juxtaposition to form ketones. Although shown on one zirconium atom, it is understood this second carboxylate moiety might in reality be a surface zirconia in close proximity to the first, the key being the proper juxtaposition for the two separate carboxylate entities to react:
ZrO(O2CR)O⁻M⁺+RCO2H→ZrO(O2CR)2+MOH 4.
ZrO(O2CR)2→ZrO(OH)(O2C—R′CH—(C═O)—R) 5.

In equation 5, the covalently coordinated zirconia carboxylates in their proper juxtaposition combine to give a derivative of the final ketone.

The zirconia carboxylate(s) in equation 5 may form the ketone products by a free radical mechanism. But if at least one of the carboxylate groups has one or more hydrogen atoms on the carbon adjacent to the carbonyl group, a carbanion mechanism leading to a more facile overall reaction is available:
ZrO(O2CR)(O2CCH2R′)+MOH→ZrO(O2CR)(O2CC⁽⁻⁾HR′)M^{(+)+H}2O 5a.
ZrO(O2CR)(O2CC⁽⁻⁾HR′)M⁽⁺⁾→ZrO(O^{(-)(O}2C-CH(RC═O)R′)M⁽⁺⁾ 5b.
ZrO(O⁽⁻⁾)(O2C—CH(RC═O)R′)M^{(+)→ZrO}2+(O═CR—C^{(-)HR′)M}⁽⁺⁾+CO2 5c.
(O═CR—C⁽⁻⁾HR′)M⁽⁺⁾+H2O→R—C(═O)CH2R′+MOH 5d.

The key reaction in equation 5b takes place by the following possible mechanism:

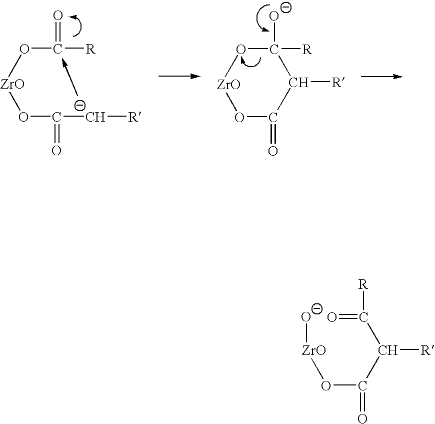

According to this mechanism the individual adsorbed carboxylate species react to give a coordinated beta carbonyl carboxylic acid. Either in its coordinated form or when the organic intermediate is released from the catalyst by reacting with the protons from fresh carboxylic acid substrates, beta carbonyl carboxylic acids readily decarboxylate under mild reaction conditions to give a ketone and carbon dioxide. In the former case the zirconia-oxy anion pulls the carbon dioxide from the organic group to form inorganic zirconia carbonate which readily loses carbon dioxide under the reaction conditions. In the latter case the free organic acid readily decarboxylates by a entropically favorable process:
ZrO(OH)(O2C—R′CH(C═O)—R)→ZrO(OH)2+R2C═O+CO2 6.

In the absence of basic promoters on the catalyst, similar mechanisms involving ketenes or concerted shifts of R groups may prevail. The free-radical or concerted mechanism takes place much more infrequently or when there is no hydrogen on the carbon atom adjacent to the carboxylate group. In this mechanism the zirconia carboxylate undergoes a homolytic cleavage to produce a zirconia-oxy radical and a carbonyl radical.

Simultaneously a different zirconia carboxylate undergoes a different homolytic cleavage to produce a zirconia radical and a carboxylate radical. Carboxylate radicals rapidly lose carbon dioxide to produce alkyl radicals which react on contact with the carbonyl radical to produce the observed ketone product. Similarly in the concerted variation on this mechanism, all electron shifts (old bonds breaking and new bonds forming) occur simultaneously without needing to form free radicals.

Evidence for a contributing radical mechanism comes from the observed production of hydrocarbon by-products (both alkanes and alkenes), carbon monoxide (from the decarbonylation of the carbonyl radical), and from observed radical rearrangement products. The relative unimportance of this mechanism is apparent from the lower reaction rates and/or the harsher conditions needed to make ketones from substrates with no hydrogens adjacent to the carboxyl group. Accompanying this more ponderous mechanism is frequently a lower yield of the ketone products with the side reactions formed from the multitude of pathways through which free radicals can decompose. And even in those cases for which all substrates have multiple hydrogen atoms adjacent to the carboxylate group, alkane, alkene, and carbon monoxide by-products, indicative of a free radical process, are produced.

Extensive prior art catalysts are known which carry out the ketonization reaction of carboxylic acids, especially those having at least one alpha hydrogen. Zirconia is also known to catalyze this reaction providing high yields at high conversions of symmetrical ketones from a single carboxylic acid. But the yields of mixed ketones is generally low owing to the discriminating nature of the catalyst toward different sized carboxylic acids.

Using the present catalyst and taking advantage of its high thermal stability, conditions have been unexpectedly discovered which overcome the natural tendency of this catalyst to form primarily symmetrical ketones even from mixtures of several carboxylic acids. The presence of the group Ia or IIa promoter enhance the mixed ketone production by lowering the lattice energy near the catalyst surface and increasing the stability of the catalytically active phases. This treatment permits using reaction temperatures allowing ketone production near statistical.

In the absence of the promoters, the temperatures at which the mixed ketones are formed become so high that the product selectivities suffer. At these extreme temperatures, the catalyst produces increasingly undesirable materials including unsaturated ketones, aldol condensation by-products, and carbon.

The unsaturated ketones form by an oxidation or dehydrogenation side-reaction. These impurities are especially troubling because general purification methods (distillation, recrystallization, and chromatography) will not remove those impurities. In fact only a separate hydrogenation reaction will.

The other by-products are generally the first of a cascade of reactions eventually culminating in carbon or coke. Not only do they destroy product, but they also eventually block the catalyst activity.

Initial experiments using groups la and IIa promoters to modify the catalytic behavior of zirconia show lower mixed ketone selectivities than using zirconia with no promoters at all. Since this process is highly endothermic, it is difficult to achieve the high sustained temperatures necessary to achieve the optimum results. Moreover higher temperatures usually lead to enhanced production of by-products as more reactive avenues open for the products already produced. Therefore it is counterintuitive that changing the conditions under which the catalyst operates would enhance this activity. And it is especially unobvious that with detrimental results at relatively lower temperatures that further raising the temperature would actually enhance the catalytic selectivity. But this is exactly what it does.

As an added bonus, treating this zirconia with the group Ia and IIa promoters does not negatively affect the ability to regenerate the catalyst. In the case of many ceramics, these promoters alter the chemistry so that surface impurities readily migrate into the bulk of the material where they become sequestered from the purgative actions of the regenerating materials. The regenerability of this catalyst is one of its positive features. And its not being affected with the chosen promoters is an unexpected and unobvious aspects.

The ability to manufacture dissymmetric ketones from two different carboxylic acids increases the range of application of this reaction substantially. It is convenient enough to prepare symmetrical ketones by this reaction. But the economic advantage in producing unsymmetrical ketones in many cases spells the difference between having a commercial route to a product and having no route. This catalyst gives an edge to a route to many of these ketones unattainable by other chemistries on a commercial scale.

This invention can be further illustrated by the following examples of preferred embodiments thereof, although it will be understood that these examples are included merely for purposes of illustration and are not intended to limit the scope of the invention. Unless otherwise indicated, all weight percentages are based on the total weight of the polymer composition and all molecular weights are weight average molecular weights. Also, all percentages are by weight unless otherwise indicated.

### EXAMPLES

The examples given below are presented only to illustrate the results possible with this invention and not to encompass the scope of applications. They include results generally in the most preferred ranges, but also examples outside of these ranges for comparison and methods for preparing the catalysts themselves. It is to be understood that these examples do not define the limits under which the catalysts will perform.

### Example 1

### Ketone Screening Reactors

The equipment for these experiments was a one inch diameter 304 stainless steel tube two feet in length heated with a Series 3210 Applied Test Systems 2 kilowatt reactor operating at temperatures of 200 to 700° C. +/−5° C. The catalyst was weighed and introduced as¼ inch diameter pellets filling about one third of the reactor topped with a 6 inch bed of 8 mm glass beads to help vaporize the liquid feed. A calibrated series 33 Harvard syringe pump was used to introduce the feed at a pre-determined rate. Screening experiments generally ran 4-8 hours to ensure a consistent product. Catalyst lifetime studies required several hundred hours of continuous operation. These experiments were aided by use of a Camille automated computer system to control the experiments.

Analyses were completed using a Varian 6890 gas chromatograph equipped with a 30 meter DB-5 capillary column and calibrated using authentic samples of the different products. The results generally agreed within 0.5 percent.

### Example 2.1

### Potassium Base Modified Zirconia Catalyst—Exchange Preparation

The charge to a 250 milliliter round bottom flask equipped with a Teflon coated stirring bar and blanketed with an inert nitrogen atmosphere throughout the reaction was 100 cubic centimeters of Norton XZ 16075 ¼inch diameter Zirconia pellets (bulk density=1.017 grams per cubic centimeter, 101.7 grams, 51 square meters per gram surface area). To this material was added sufficient 10 weight percent aqueous potassium hydroxide solution to just cover the pellets (75 milliliters solution). Immediately after mixing the temperature of the mixture rose to 450° C. but quickly subsided thereafter. To ensure complete contact, a vacuum (40 millimeters mercury) was drawn on the mixture followed by its release through admitted nitrogen a total of three times. After the last vacuum treatment, the two components were allowed to stand together for 48 hours catalyst before workup.

The workup consisted of decanting the spent potassium hydroxide solution and washing the residual catalyst till the washings were no longer basic. This treatment generally required successive treatments with 4×75 milliliter quantities of deionized water. The treated catalyst was then dried for two days in a stream of dry nitrogen followed by heating to 200° C. for 4 hours in an oven. A small amount (ca. 0.5 grams) of catalyst fines were discarded with the base treatment. The yield of dried product was 102.7 grams. Elemental analysis showed the incorporation of 1.19 weight percent potassium into the catalyst matrix.

### Example 2.2

### Calcium Base Modified Zirconia Catalyst—Incipient Wetness Preparation

The charge to the 250 milliliter round bottom flask equipped with a Teflon coated stirring bar and blanketed with nitrogen was 100 milliliters of Norton XZ 16075 ¼ inch diameter pellets (bulk density=1.017. 101.7 grams, surface area=51 square meters per gram). To this slowly rotating material was added dropwise a 10.1 weight percent solution of calcium acetate in water (0.67 M). This treatment continued till the solid material would absorb no more solution and there was evidence of liquid beginning to appear in the bottom of the flask. This treatment required 46.5 milliliters of the solution. The total calcium acetate incorporated was 4.93 grams.

The workup consisted of removing as much water as possible using a rotary evaporator operating at 10 millimeters mercury vacuum and at a temperature ramping up to 100° C. over two hours. This treatment removed 37.0 milliliters of water. The residual water was removed in a stream of dry nitrogen over 24 hours followed by heating for 24 hours in a vacuum oven at 200° C. and 50 millimeters mercury pressure. Then this material was calcined at 450° C. for 4 hours till all traces of carbon had disappeared.

The final catalyst amounted to 104.7 grams. Elemental analysis revealed a calcium content of 1.16 weight percent.

### Example 2.3

### Sodium Base Modified Zirconia Catalyst—Coprecipitation Preparation

The material added to a 1-liter round bottom flask equipped with an overhead stirrer, a 500 milliliter addition funnel, a reflux condenser, and a thermowell containing a 250° C. thermometer was 400 milliliters of 8.25 weight percent sodium hydroxide (2.23 M, 0.892 mole). To this well-stirred solution added in small portions through a powder funnel was 100.2 grams of zirconyl chloride octahydrate (0.310 gram atom). The solution warmed during the addition and when it was complete the contents were heated to reflux for 2 hours to ensure complete reaction.

Workup consisted of filtering the white mass through a sintered glass filter and washing the filter cake with deionized water till no more chloride was detected in the filtrate. The total wash amounted to 2.5 liters. Then the filter cake was dried overnight in a stream of dry nitrogen and finally in a vacuum oven at 200° C. for 6 hours.

The total product amounted to 47.9 grams material. Elemental analysis showed a sodium content of 0.93 percent. This material was broken into small pieces and sieved. Material collected at 4-10 mesh was used for the catalyst studies. The remainder of the material was reprocessed with additional batches of precipitation prepared zirconia. The total sieved material for the catalyst studies amounted to 140 cubic centimeters with a bulk density of 1.07 grams per cubic centimeter.

### Example 3

### Unpromoted Zirconia Catalyst—Preparation of Diethyl Ketone

The charge to the ketone screening reactor was 70 cubic centimeters of unpromoted Norton XZ 16075 ¼ inch diameter pellets (bulk density =1.017, 71.2 grams) topped with a nine inch bed of 8 millimeter glass beads to serve as a substrate preheater. The catalyst bed itself was positioned near the middle of the reactor. The catalyst was heated to 425° C. with a nitrogen purge of 125 cubic centimeters per minute. This purge was continued till the substrate feed began.

The feed to the reactor was a solution of 90.0 weight percent propionic acid and 10.0 weight percent water. The water served as a heat transfer agent helping keep the temperature uniform throughout the catalyst during the reaction. The feed rate was 70+/−2 cubic centimeters per hour so that the calculated space velocity was 1.0 volume substrate per volume catalyst per hour. The feed time amounted to 4.1 hours during which time a total of 288 milliliters of substrate (d=0.993, 285.9 grams total, 257.3 grams propionic acid, 3.47 moles) was fed and the temperature range was 390-430° C.

Gas chromatographic analysis of the final product showed the following results: The recovered propionic acid amounted to 13.8 grams giving an acid conversion of 94.6 percent. And the 3-pentanone amounted to 136.1 grams (1.58 moles) giving a selectivity of 95.2 percent. The calculated production rate of 3-pentanone was 29.6 pounds per cubic foot catalyst per hour.

The purpose of this experiment was to show the results of using unpromoted zirconia catalyst to prepare a symmetrical ketone.

### Example 4.1

### Unpromoted Zirconia Catalyst—Preparation of Methyl Isopropyl Ketone

The charge to the ketone screening reactor was 74 cubic centimeters of the unpromoted zirconia catalyst described in example 3 topped with a nine inch bed of 8 millimeter glass beads to serve as a substrate preheater. The bottom of the catalyst bed extended to the middle of the reactor. The bed was heated to 425° C. with a nitrogen purge of 125 cubic centimeters per minute. This purge was continued till the substrate feed began.

The feed to the reactor was a solution containing 45.5 weight percent acetic acid, 44.5 weight percent isobutyric acid, and 10.0 weight percent water. The molar ratio of acetic to isobutyric acid was 1.5:1. The presence of water served as a heat transfer agent helping to keep the temperature uniform throughout the catalyst bed during the reaction. The feed rate was 74+/−2 cubic centimeters per hour so that the calculated space velocity was 1.0 volume substrate per volume catalyst per hour. The feed time amounted to 4.0 hours during which time a total of 295 milliliters of substrate (d=1.000, 295.1 grams total; 134.3 grams acetic acid, 2.24 moles; 131.3 grams isobutyric acid, 1.49 moles) was fed and the temperature range was 395-430° C.

Gas chromatographic analysis of the final product showed the following results: The acetic acid recovered amounted to 0.3 grams giving an acetic acid conversion of 99.8 percent. The isobutyric acid recovered amounted to 5.6 grams giving an isobutyric acid conversion of 95.7 percent. And the yields of the different ketone products were as follows: acetone (36.3 grams), methyl isopropyl ketone (77.0 grams), methyl isopropenyl ketone (0.1 gram), diisopropyl ketone (29.4 grams). The calculated production rate of methyl isopropyl ketone was 16.2 pounds per cubic foot catalyst per hour.

Based on these numbers the selectivities to these products were as follows: acetone (56.0 percent, based on acetic acid consumed); methyl isopropyl ketone (40.1 percent, based on acetic acid consumed; 62.7 percent, based on isobutyric acid consumed); methyl isopropenyl ketone (0.1 percent, based on acetic acid consumed; 0.1 percent, based on isobutyric acid consumed); and, diisopropyl ketone (36.1 percent, based on isobutyric acid consumed).

The corresponding selectivities expected statistically with this feed ratio are as follows: acetone (42.9 percent based on acetic acid), methyl isopropyl ketone (57.1 percent based on acetic acid, 75.0 percent based on isobutyric acid), diisopropyl ketone (25.0 percent based on isobutyric acid).

The purpose of this experiment was to show the results of using unpromoted zirconia catalyst to prepare an unsymmetrical ketone.

### Example 4.2

### Unpromoted Zirconia Catalyst—Preparation of Methyl Isopropyl Ketone

Experiment 4.1 was repeated except the reactor temperature was raised to 475° C.

The feed to the reactor was a solution containing 45.5 weight percent acetic acid, 44.5 weight percent isobutyric acid, and 10.0 weight percent water. The molar ratio of acetic to isobutyric acid was 1.5:1. The presence of water served as a heat transfer agent helping to keep the temperature uniform throughout the catalyst bed during the reaction. The feed rate was 74+/−2 cubic centimeters per hour so that the calculated space velocity was 1.0 volume substrate per volume catalyst per hour. The feed time amounted to 3.9 hours during which time a total of 292 milliliters of substrate (d=1.000, 291.8 grams total; 132.8 grams acetic acid, 2.21 moles; 129.9 grams isobutyric acid, 1.47 moles) was fed and the temperature range was 465-485° C.

Gas chromatographic analysis of the final product showed the following results: The acetic acid recovered amounted to 0.0 grams giving an acetic acid conversion of 100.0 percent. The isobutyric acid recovered amounted to 1.6 grams giving an isobutyric acid conversion of 98.8 percent. And the yields of the different ketone products were as follows: acetone (35.5 grams), methyl isopropyl ketone (76.2 grams), methyl isopropenyl ketone (0.3 gram), diisopropyl ketone (29.7 grams). The calculated production rate of methyl isopropyl ketone was 16.5 pounds per cubic foot catalyst per hour.

Based on these numbers the selectivities to these products were as follows: acetone (55.3 percent, based on acetic acid consumed); methyl isopropyl ketone (40.0 percent, based on acetic acid consumed; 60.8 percent, based on isobutyric acid consumed); methyl isopropenyl ketone (0.2 percent, based on acetic acid consumed; 0.2 percent, based on the isobutyric acid consumed); and diisopropyl ketone (35.7 percent, based on the isobutyric acid consumed).

The purpose of this experiment was to show the similarity in results in using unpromoted zirconia catalyst to prepare an unsymmetrical ketone at a higher temperature.

### Example 5.1

### Potassium Promoted Zirconia Catalyst—Preparation of Methyl Isopropyl Ketone

The charge to the ketone screening reactor was 75 cubic centimeters of the potassium promoted zirconia catalyst whose preparation was described in example 2.1 topped with a nine inch bed of 8 millimeter glass beads to serve as a substrate preheater. The catalyst bed was positioned near the middle of the reactor. The bed was heated to 425° C. with a nitrogen purge of 125 cubic centimeters per minute. This purge was continued till the substrate feed began.

The feed to the reactor was a solution containing 45.5 weight percent acetic acid, 44.5 weight percent isobutyric acid, and 10.0 weight percent water. The molar ratio of acetic to isobutyric acid was 1.5:1. The presence of water served as a heat transfer agent helping to keep the temperature uniform throughout the catalyst bed during the reaction. The feed rate was 75+/−2 cubic centimeters per hour so that the calculated space velocity was 1.0 volume substrate per volume catalyst per hour. The feed time amounted to 4.0 hours during which time a total of 299 milliliters of substrate (d=1.000, 299.0 grams total; 136.0 grams acetic acid, 2.27 moles; 133.0 grams isobutyric acid, 1.51 moles) was fed and the temperature range was 405-440° C.

Gas chromatographic analysis of the final product showed the following results: The acetic acid recovered amounted to 0.2 grams giving an acetic acid conversion of 99.9 percent. The isobutyric acid recovered amounted to 5.8 grams giving an isobutyric acid conversion of 95.6 percent. And the yields of the different ketone products were as follows: acetone (39.1 grams), methyl isopropyl ketone (73.8 grams), methyl isopropenyl ketone (0.1 gram), diisopropyl ketone (32.7 grams). The calculated production rate of methyl isopropyl ketone was 15.4 pounds per cubic foot catalyst per hour.

Based on these numbers the selectivities to these products were as follows: acetone (59.5 percent, based on acetic acid consumed); methyl isopropyl ketone (37.9 percent, based on acetic acid consumed; 59.4 percent, based on isobutyric acid consumed); methyl isopropenyl ketone (0.1 percent, based on acetic acid consumed; 0.1 percent, based on the isobutyric acid consumed); and diisopropyl ketone (39.7 percent, based on the isobutyric acid consumed).

The purpose of this experiment was to show the results of using potassium promoted zirconia catalyst to prepare an unsymmetrical ketone at a temperature below optimum.

### Example 5.2

### Potassium Promoted Zirconia Catalyst—Preparation of Methyl Isopropyl Ketone

Experiment 5.1 was repeated except the reaction temperature was raised to 475° C.

The feed to the reactor was a solution containing 45.5 weight percent acetic acid, 44.5 weight percent isobutyric acid, and 10.0 weight percent water. The molar ratio of acetic to isobutyric acid was 1.5:1. The presence of water served as a heat transfer agent helping to keep the temperature uniform throughout the catalyst bed during the reaction. The feed rate was 75+/−2 cubic centimeters per hour so that the calculated space velocity was 1.0 volume substrate per volume catalyst per hour. The feed time amounted to 4.0 hours during which time a total of 302 milliliters of substrate (d=1.000, 301.8 grams total; 137.3 grams acetic acid, 2.29 moles; 134.3 grams isobutyric acid, 1.52 moles) was fed and the temperature range was 465-490° C.

Gas chromatographic analysis of the final product showed the following results: The acetic acid recovered amounted to 0.0 grams giving an acetic acid conversion of 100.0 percent. The isobutyric acid recovered amounted to 1.8 grams giving an isobutyric acid conversion of 98.7 percent. And the yields of the different ketone products were as follows: acetone (34.2 grams), methyl isopropyl ketone (86.6 grams), methyl isopropenyl ketone (0.2 gram), diisopropyl ketone (27.4 grams). The calculated production rate of methyl isopropyl ketone was 18.0 pounds per cubic foot catalyst per hour.

Based on these numbers the selectivities to these products were as follows: acetone (51.5 percent, based on acetic acid consumed); methyl isopropyl ketone (44.0 percent, based on acetic acid consumed; 66.9 percent, based on isobutyric acid consumed); methyl isopropenyl ketone (0.1 percent, based on acetic acid consumed; 0.2 percent, based on the isobutyric acid consumed); and diisopropyl ketone (31.9 percent, based on the isobutyric acid consumed).

The purpose of this experiment was to show the improvement in using potassium promoted zirconia catalyst to prepare an unsymmetrical ketone at a higher, optimum temperature.

### Example 6

### Calcium Promoted Zirconia Catalyst—Preparation of Methyl Isopropyl Ketone

The charge to the ketone screening reactor was 74 cubic centimeters of the calcium promoted zirconia catalyst whose preparation was described in example 2.2 topped with a nine inch bed of 8 millimeter glass beads to serve as a substrate preheater. The catalyst bed was positioned near the middle of the reactor. The bed was heated to 475° C. with a nitrogen purge of 125 cubic centimeters per minute. This purge was continued till the substrate feed began.

The feed to the reactor was a solution containing 45.5 weight percent acetic acid, 44.5 weight percent isobutyric acid, and 10.0 weight percent water. The molar ratio of acetic to isobutyric acid was 1.5:1. The presence of water served as a heat transfer agent helping to keep the temperature uniform throughout the catalyst bed during the reaction. The feed rate was 74+/−2 cubic centimeters per hour so that the calculated space velocity was 1.0 volume substrate per volume catalyst per hour. The feed time amounted to 4.1 hours during which time a total of 300 milliliters of substrate (d=1.000, 299.7 grams total; 136.4 grams acetic acid, 2.35 moles; 133.4 grams isobutyric acid, 1.55 moles) was fed and the temperature range was 465-490° C.

Gas chromatographic analysis of the final product showed the following results: The acetic acid recovered amounted to 0.0 grams giving an acetic acid conversion of 100.0 percent. The isobutyric acid recovered amounted to 8.6 grams giving an isobutyric acid conversion of 93.6 percent. And the yields of the different ketone products were as follows: acetone (39.1 grams), methyl isopropyl ketone (79.4 grams), methyl isopropenyl ketone (0.2 gram), diisopropyl ketone (28.7 grams). The calculated production rate of methyl isopropyl ketone was 16.1 pounds per cubic foot catalyst per hour.

Based on these numbers the selectivities to these products were as follows: acetone (57.3 percent, based on acetic acid consumed); methyl isopropyl ketone (39.3 percent, based on acetic acid consumed; 63.6 percent, based on isobutyric acid consumed); methyl isopropenyl ketone (0.1 percent, based on acetic acid consumed; 0.2 percent, based on the isobutyric acid consumed); and diisopropyl ketone (34.7 percent, based on the isobutyric acid consumed).

The purpose of this experiment was to show the results of using calcium promoted zirconia catalyst to prepare an unsymmetrical ketone.

### Example 7

### Sodium Promoted Zirconia Catalyst—Preparation of Methyl Isopropyl Ketone

The charge to the ketone screening reactor was 75 cubic centimeters of the sodium promoted zirconia catalyst whose preparation was described in Example 2.3 topped with a nine inch bed of 8 millimeter glass beads to serve as a substrate preheater. The catalyst bed was positioned near the middle of the reactor. The bed was heated to 475° C. with a nitrogen purge of 125 cubic centimeters per minute. This purge was continued till the substrate feed began.

The feed to the reactor was a solution containing 45.5 weight percent acetic acid, 44.5 weight percent isobutyric acid, and 10.0 weight percent water. The molar ratio of acetic to isobutyric acid was 1.5:1. The presence of water served as a heat transfer agent helping to keep the temperature uniform throughout the catalyst bed during the reaction. The feed rate was 75+/−2 cubic centimeters per hour so that the calculated space velocity was 1.0 volume substrate per volume catalyst per hour. The feed time amounted to 4.3 hours during which time a total of 320 milliliters of substrate (d=1.000, 320.4 grams total; 145.8 grams acetic acid, 2.43 moles; 142.6 grams isobutyric acid, 1.62 moles) was fed and the temperature range was 460-485° C.

Gas chromatographic analysis of the final product showed the following results: The acetic acid recovered amounted to 0.0 grams giving an acetic acid conversion of 100.0 percent. The isobutyric acid recovered amounted to 5.6 grams giving an isobutyric acid conversion of 96.1 percent. And the yields of the different ketone products were as follows: acetone (38.0 grams), methyl isopropyl ketone (88.2 grams), methyl isopropenyl ketone (0.2 gram), diisopropyl ketone (29.4 grams). The calculated production rate of methyl isopropyl ketone was 17.1 pounds per cubic foot catalyst per hour.

Based on these numbers the selectivities to these products were as follows: acetone (53.9 percent, based on acetic acid consumed); methyl isopropyl ketone (42.2 percent, based on acetic acid consumed; 65.9 percent, based on isobutyric acid consumed); methyl isopropenyl ketone (0.1 percent, based on acetic acid consumed; 0.2 percent, based on the isobutyric acid consumed); and diisopropyl ketone (33.1 percent, based on the isobutyric acid consumed).

The purpose of this experiment was to show the results of using sodium promoted zirconia catalyst to prepare an unsymmetrical ketone.

### Example 8

### Potassium Promoted Zirconia Catalyst—Lifetime Studies/Regeneration

Experiment 5.2 was extended to determine the lifetime of its potassium promoted zirconia catalyst. Over the course of 36 days the catalyst remained active. During this time the methyl isopropyl ketone selectivity based on the isobutyric acid fed averaged 66.2 percent. At the end of the experiment, its deactivation was signaled when the product selectivity dipped abruptly by one-third to 42.0 percent.

At this time the catalyst was removed from the reactor for inspection. From an initial charge of 76.6 grams (density=1.021, 75 cubic centimeters) of pure white material, the recovered material amounted to 79.9 grams with a dark coating of carbon evident throughout the catalyst pellets.

This material was placed in a muffle furnace and heated to 400° C. for 6 hours in air. At the end of this time the recovered product was off-white and weighed 74.9 grams. The dark coloration throughout the catalyst pellets had disappeared.

After sieving to remove fines, 72 cubic centimeters of regenerated catalyst was reintroduced into into the reactor and the lifetime study was resumed. Over the next 31 days, the methyl isopropyl ketone selectivity based on isobutyric acid fed averaged 63.8 percent.

The purpose of this experiment was to determine how well the catalyst performed after regeneration.

### Example 9

### Unpromoted Titania Catalyst—Preparation of Methyl Isopropyl Ketone

The charge to the ketone screening reactor was 75 cubic centimeters of ¼ inch diameter pellets of Norton XT 25376 Anatase titania catalyst (density=0.833, 62.5 grams) whose surface area was 168 square meters per gram topped with a nine inch bed of 8 millimeter glass beads to serve as a substrate preheater. The catalyst bed was positioned near the middle of the reactor. The bed was heated to 475° C. with a nitrogen purge of 125 cubic centimeters per minute. This purge was continued till the substrate feed began.

The feed to the reactor was a solution containing 45.5 weight percent acetic acid, 44.5 weight percent isobutyric acid, and 10.0 weight percent water. The molar ratio of acetic to isobutyric acid was 1.5:1. The presence of water served as a heat transfer agent helping to keep the temperature uniform throughout the catalyst bed during the reaction. The feed rate was 75+/−2 cubic centimeters per hour so that the calculated space velocity was 1.0 volume substrate per volume catalyst per hour. The feed time amounted to 4.0 hours during which time a total of 296 milliliters of substrate (d=1.000, 295.7 grams total; 134.5 grams acetic acid, 2.24 moles; 131.6 grams isobutyric acid, 1.49 moles) was fed and the temperature range was 460-480° C.

Gas chromatographic analysis of the final product showed the following results: The acetic acid recovered amounted to 0.1 grams giving an acetic acid conversion of 99.9 percent. The isobutyric acid recovered amounted to 1.2 grams giving an isobutyric acid conversion of 99.1 percent. And the yields of the different ketone products were as follows: acetone (35.0 grams), methyl isopropyl ketone (74.9 grams), methyl isopropenyl ketone (3.4 gram), diisopropyl ketone (30.7 grams). The calculated production rate of methyl isopropyl ketone was 15.6 pounds per cubic foot per hour.

Based on these numbers the selectivities to these products were as follows: acetone (53.8 percent, based on acetic acid consumed); methyl isopropyl ketone (38.8 percent, based on acetic acid consumed; 58.8 percent, based on isobutyric acid consumed); methyl isopropenyl ketone (1.8 percent, based on acetic acid consumed; 2.7 percent, based on the isobutyric acid consumed); and diisopropyl ketone (36.3 percent, based on the isobutyric acid consumed).

The purpose of this experiment was to compare the results of the zirconia catalyst to prepare an unsymmetrical ketone with other Group IVB catalysts especially with respect to the dehydrogenated byproducts containing the isopropenyl group.

The invention has been described in detail with particular reference to preferred embodiments thereof, but it will be understood that variations and modifications can be effected within the spirit and scope of the invention.

We claim:

1. A process for preparing unsymmetrical ketones, comprising contacting at least two different carboxylic acids with a catalyst comprising zirconia and a Group 1 or Group 2 metal promoter, at a temperature of 450 to 700° C.

2. The process according to claim 1, wherein the temperature is 450 to 600° C.

3. The process according to claim 1, wherein the temperature is 450 to 500° C.

4. The process according to claim 1, wherein the Group 1 metal promoter is selected from the group consisting of lithium, sodium, potassium, rubidium, and cesium.

5. The process according to claim 1, wherein the Group 2 metal promoter is selected from the group consisting of beryllium, magnesium, calcium, strontium, and barium.

6. The process according to claim 1, wherein the Group 1 or 2 metal promoter is selected from the group consisting of potassium, calcium, and sodium.

7. The process according to claim 1, wherein the catalyst comprises 0.01 to 10 weight percent of the Group 1 or 2 metal promoter.

8. The process according to claim 1, wherein the carboxylic acids are fed into a reactor at a rate of 0.1 to 10 volumes of liquid feed per volume of catalyst per hour.

9. The process according to claim 1, wherein the carboxylic acids are fed into a reactor at a rate of 0.5 to 5 volumes of liquid feed per volume of catalyst per hour.

10. The process according to claim 1, wherein the at least two different carboxylic acids are fed into a reactor at a molar ratio 4:1 to 1:4.

11. The process according to claim 1, wherein the at least two different carboxylic acids are fed into a reactor at a molar ratio 2:1 to 1:2.

12. A process for preparing methyl isopropyl ketone, comprising contacting acetic acid and isobutyric acid, at a temperature of 450 to 700° C., with a catalyst comprising zirconia treated with KOH.
